(19) United States
(12) Patent Application Publication
(10) Pub. No.: US 2022/0387801 A1
(43) Pub. Date: Dec. 8, 2022

(54) SYSTEMS AND METHODS UTILIZING PHASE-BASED BIOMARKER IN THE BRAIN

(71) Applicant: Duke University, Durham, NC (US)

(72) Inventors: Stephen Schmidt, Durham, NC (US); Warren Grill, Durham, NC (US)

(21) Appl. No.: 17/829,449

(22) Filed: Jun. 1, 2022

Related U.S. Application Data
(60) Provisional application No. 63/195,780, filed on Jun. 2, 2021.

(51) Int. Cl. A61N 1/36

(52) U.S. Cl. CPC A61N 1/36135; A61N 1/36146; A61N 1/0534

(57) ABSTRACT

Embodiments of the present disclosure include a method for acquiring, with a device, a plurality of electrical signals from a brain of a subject, and determining whether a biomarker is present in the plurality of electrical signals. The biomarker comprises at least one phase metric associated with the electrical signal, and further includes adjusting at least one therapy parameter based on biomarker assessment.

### RELATED APPLICATIONS

This application claims priority to and the benefit of U.S. Provisional Patent Application No. 63/195,780 filed Jun. 2, 2021, which is incorporated herein by reference in its entirety for all purposes.

### GOVERNMENT FUNDING

This invention was made with Government support under Federal Grant No. UH3 NS103468 awarded by the National Institutes of Health. The Federal Government has certain rights to the invention.

### FIELD

The present disclosure provides systems and methods utilizing a phase-based biomarker in the brain to identify and/or treat various diseases associated with pathological neural activity.

### BACKGROUND

Nervous system disorders affect millions of people, causing a degradation of life, and in some cases, death. Nervous system disorders may include disorders of the central nervous system and peripheral nervous system. Some nervous system disorders may be considered “neurological movement disorders,” and may include, for example without limitation, epilepsy, Parkinson's disease, essential tremor, dystonia, and multiple sclerosis (MS). Neurological movement disorders may be characterized by periods of involuntary movements and/or loss of muscle control.

As an example of a neurological movement disorder, Parkinson's Disease (PD) is generally characterized by poverty and slowness of movement (akinesia and bradykinesia), muscle stiffness (rigidity), tremor at rest, and gait and balance abnormalities that may lead to an inability to perform normal daily life activities. Some patients suffering from neurological movement disorders may also develop symptoms called dyskinesias and motor fluctuations, which may be side effects of certain anti-Parkinson's medication. It is believed that PD is caused by the degeneration of dopaminergic neurons in the substantia nigra pars compacta, a brain structure of the basal ganglia involved in the control of movement. The loss of dopamine in the basal ganglia is believed to secondarily cause a cascade of abnormal activity in the other nuclei of the basal ganglia, thalamus and cortex. This has been detected in animals and humans as changes in neuronal firing patterns, firing frequencies, and in the tendency of these neurons to fire in an oscillatory manner. These abnormal oscillations and firing patterns are thought to underlie the classic motor symptoms of PD and have been shown to be reversible with the dopamine medication used to treat PD.

There are various approaches in treating nervous system disorders, such as neurological movement disorders. Treatment therapies can include any number of possible modalities alone or in combination including, for example, electrical stimulation, magnetic stimulation, drug infusion, and/or brain temperature control. Each of these treatment modalities may be employed using closed-loop feedback control. Such closed-loop feedback control techniques may control stimulation based on received neurological signals (e.g., from a monitoring element) carrying information about a symptom or a condition of a nervous system disorder. Such a neurological signal can include, for example, electrical signals (such as local field potentials (LFPs), electroencephalogram (EEG), electrocorticogram (ECoG), and/or electrocardiogram (EKG) signals), chemical signals, and/or other types of biological signals (such as changes in the quantity of neurotransmitters).

### SUMMARY

Embodiments of the present disclosure include a method that involves acquiring, with a device, a plurality of electrical signals from a brain of a subject, wherein the plurality of electrical signals includes a first electrical signal acquired from a first brain region and a second electrical signal acquired from a second brain region. The method further includes determining whether a biomarker is present in the plurality of electrical signals, wherein the biomarker comprises at least one phase metric associated with the first electrical signal and the second electrical signal. The method further includes adjusting at least one therapy parameter based on the presence of the biomarker.

In some embodiments, the first brain region is a subthalamic nucleus, an internal segment of the globus pallidus, a motor cortex, a thalamus, an external segment of the globus pallidus, a striatum, or a cerebellum and the second brain region is a subthalamic nucleus, an internal segment of the globus pallidus, a motor cortex, a thalamus, an external segment of the globus pallidus, a striatum, or a cerebellum; wherein the second brain region is different than the first brain region.

In some embodiments, the phase metric is a phase locking value.

In some embodiments, the phase locking value includes a locked phase and a magnitude.

In some embodiments, the method includes determining the phase metric by temporal windowing and frequency windowing the first electrical signal and the second electrical signal.

In some embodiments, the method includes determining whether the biomarker is present includes frequency isolation of the plurality of electrical signals.

In some embodiments, the frequency isolation of the plurality of electrical signals includes a bandpass filter with a range of 13 Hz to 30 Hz.

In some embodiments, the method includes detecting a synchronous event including determining a baseline locked phase range and comparing the locked phase to the baseline locked phase range.

In some embodiments, the method includes determining a time duration of the synchronous event; and determining a total number of synchronous events.

In some embodiments, the therapy parameter relates to an electrical stimulation to the brain by the device.

In some embodiments, the therapy parameter is an amplitude of electrical stimulation, a pulse duration of the electrical stimulation, a frequency of electrical stimulation, a timing of the electrical stimulation, or a temporal pattern of the electrical stimulation.

In some embodiments, the method includes adjusting at least one therapy parameter based on the presence of the biomarker includes closed-loop control of the biomarker.

In some embodiments, the therapy parameter is a medication dose.

Embodiments of the present disclosure also include a system comprising a device including a processor, and a plurality of electrodes in communication with the device. The plurality of electrodes are configured to be implanted in a brain of a subject. The processor is configured to acquire, via the plurality of electrodes, a plurality of electrical signals, wherein the plurality of electrical signals includes a first electrical signal acquired from a first brain region and a second electrical signal acquired from a second brain region. The processor is also configured to determine whether a biomarker is present in the plurality of electrical signals, wherein the biomarker comprises at least one phase metric associated with the first electrical signal and the second electrical signal. The processor is also configured to adjust at least one therapy parameter based on the presence of the biomarker.

In some embodiments, the device further includes a pulse generator electrically coupled to the plurality of electrodes, wherein the pulse generator includes a power source.

In some embodiments, the device is implantable.

In some embodiments, the phase metric is a phase locking value including a locked phase and a magnitude.

In some embodiments, the therapy parameter relates to an electrical stimulation to the brain delivered by the plurality of electrodes.

In some embodiments, the processor adjusts the at least one therapy parameter based on closed-loop control of the biomarker.

In some embodiments, the processor is configured to detect a synchronous event by determining a baseline locked phase range and comparing the locked phase to the baseline locked phase range, and the processor is configured to detect a time duration of the synchronous event and determine a total number of synchronous events.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram illustrating a deep brain stimulation (DBS) system used to perform the methods of the present disclosure.

FIG. 2 is a block diagram illustrating components of a medical device used to perform the methods of the present disclosure.

FIG. 3 is a flow chart illustrating a method of controlling PD symptoms in a patient, according to one embodiment of the present disclosure.

FIG. 4 illustrates a method of estimating phase from simultaneous local field potential measurements including two preprocessing steps, according to one embodiment of the present disclosure.

FIG. 5 illustrates two example phase locking value calculations, according to one embodiment of the present disclosure.

FIG. 6 is an example of calculation of a phase metric, according to one embodiment of the disclosure.

FIG. 7A is an example closed-loop control diagram, according to one embodiment of the present disclosure.

FIG. 7B is an example control scheme utilizing a recurrent neural network, according to one embodiment of the present disclosure.

FIG. 8 includes radial histograms of all phase lags between the STN and GP within a hemisphere (left) or between STNs (right).

FIG. 9 includes intra-hemispheric phase locking value and locked phase in 10 second windows for untreated (blue) and treated (orange, therapeutic DBS) states.

FIG. 10 includes bar graphs showing deep brain stimulation reduces time spent in a synchronized event. The total time spent in synchronized events is calculated for untreated (blue, DBS off) and treated (orange, therapeutic DBS on) state. In each case application of therapeutic DBS reduces time spent synchronized.

FIG. 11 includes line graphs showing deep brain stimulation reduced the duration of synchronous events in the presence of pathological synchronization. The frequency of synchronous events by duration for intra-hemispheric (top) and STN-STN coherence (bottom).

FIG. 12 illustrates a correlation between bradykinesia and phase locking value. (Left) A high degree of correlation between bradykinesia (measured by hand grasp speed) and phase locking value between the STN and GP of the same hemisphere during the hand grasps. (Right) Additional correlation between bradykinesia and inter-hemispheric phase locking value of both GPs.

FIG. 13 illustrates that phase locking value is negatively corelated to deep brain stimulation amplitude. (Top) Intra-hemispheric phase locking value for each quintile of the deep brain stimulation amplitude (100% corresponds to clinical value) for the left and right hemispheres, respectively. As deep brain stimulation amplitude increases the phase locking value decreases. (Bottom) Phase locking value and bradykinesia correlations for the same.

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

“Phase locking value” (PLV) is used herein to describe a measure of the phase synchrony between two time-series. The phase locking value may include a locked phase and a magnitude. The PLV magnitude is a length of an average vector of all phases. The PLV locked phase is an angle of the resultant vector. In other words, PLV magnitude is a metric of how synchronous the signals are and the PLV locked phase is a measure of the lead/lag between the two.

“Synchronous event” is used herein to describe continuous periods of having a locked phase within a range surrounding a common or pre-determined phase lag.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Deep Brain Stimulation

Embodiments of the present disclosure include systems and methods for utilizing a phase-based biomarker in the brain to identify and/or treat various diseases associated with pathological neural activity. In accordance with these embodiments, a parameter of measured brain activity, such as a biomarker, may be defined from measured local field potential (LFP) signals from the patient's motor cortex and STN. The biomarkers may be monitored by an implantable medical device (IMD) or external programmer or controller. The biomarkers may be used to assess the patient's current disease state. The biomarker may also be used to serve as an indicator of therapy effectiveness in a device or system. Further, the biomarker may provide feedback to control the IMD. In some examples, information regarding one or more patient specific biomarkers may allow for an enhanced ability to provide individualized therapy.

Certain examples consistent with the present disclosure include an implantable medical device and/or lead system adapted to electrically stimulate targets in the brain to modulate one or more biomarkers indicative of PD. The IMD may continually adjust one or more therapy parameters to maintain certain biomarkers, and suppress others that indicate the patient is within a therapeutic window which provide optimum symptom control with minimal side-effects. In some examples, a medical device may control the delivery of stimulation based the presence of one or more biomarkers in either the beta or gamma frequency ranges.

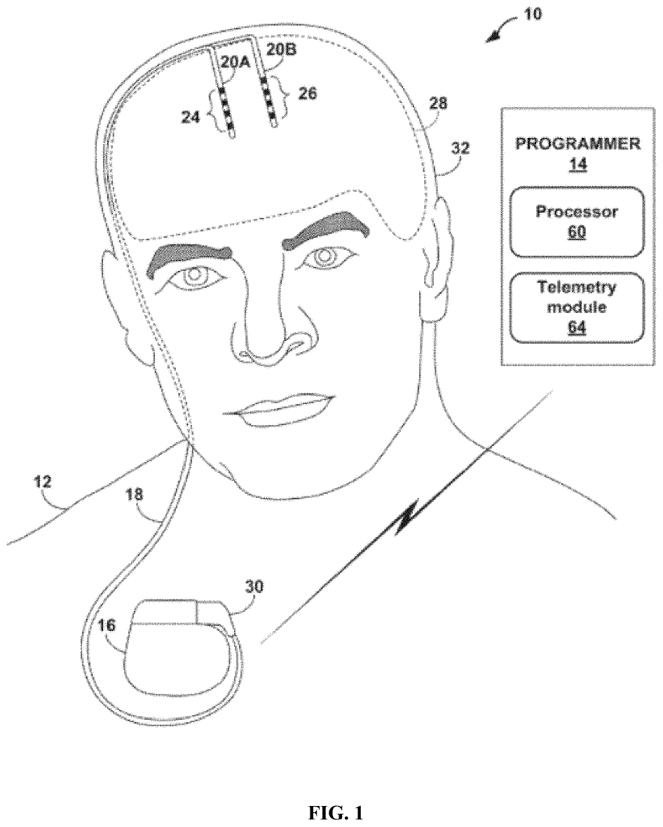

FIG. 1 is a diagram illustrating an example deep brain stimulation (DBS) system that may be used to implement the techniques of this disclosure. In FIG. 1, example therapy system 10 may deliver electrical stimulation therapy to control a patient condition, such as a movement disorder or a neurodegenerative impairment of patient 12. In one embodiment, the patient 12 is a human patient. In some cases, however, therapy system 10 may be applied to other mammalian or non-mammalian non-human patients. While movement disorders and neurodegenerative impairment are primarily referred to in this disclosure, in other examples, therapy system 10 may provide therapy to manage symptoms of other patient conditions, such as, but not limited to, seizure disorders or psychological disorders.

A movement disorder or other neurodegenerative impairment may include symptoms such as, for example, muscle control impairment, motion impairment or other movement problems, such as rigidity, bradykinesia, rhythmic hyperkinesia, non-rhythmic hyperkinesia, and akinesia. In some cases, the movement disorder may be a symptom of PD. However, the movement disorder may be attributable to other patient conditions. Although PD is primarily referred to throughout the remainder of the disclosure, the therapy systems and methods described herein may also be useful for controlling symptoms of other conditions, such as other movement disorders or neurodegenerative impairment.

In the example of FIG. 1, therapy system 10 includes a medical device programmer 14, an implantable medical device (IMD) 16, a lead extension 18, and leads 20A and 20B with respective sets of electrodes 24, 26. In some examples, therapy system 10 may include one or more additional medical devices, which may also be in communication with the medical device programmer 14. In the example shown in FIG. 1, electrodes 24, 26 of leads 20A, 20B are positioned to sense LFPs and/or deliver electrical stimulation to a tissue site within brain 28, such as a deep brain site under the dura mater of brain 28 of patient 12. In some examples, delivery of stimulation to one or more regions of brain 28, such as the subthalamic nucleus (STN), globus pallidus internus (GPi), motor cortex such as M1, or thalamus, may be an effective treatment to manage movement disorders, such as Parkinson's Disease or essential tremor.

IMD 16 includes a therapy module that includes a stimulation generator that generates and delivers electrical stimulation therapy to patient 12 via a subset of electrodes 24, 26 of leads 20A and 20B, respectively. The subset of electrodes 24, 26 that are used to deliver electrical stimulation to patient 12, and, in some cases, the polarity of the subset of electrodes 24, 26, may be referred to as a stimulation electrode combination. Using the techniques described in this disclosure, a subset of electrodes 24, 26 of leads 20A and 20B, respectively, may be used to deliver electrical stimulation to patient 12.

For example, electrodes 24, 26 of leads 20A and 20B, respectively, may be used to deliver electrical stimulation to patient 12 at a frequency shown to affect the individual patient's biomarkers. In some examples, electrical stimulation is provided in a biphasic manner. For example, stimulation may be provided at a particular frequency at a voltage that alternates between +2V and −2V.

In another example, the frequency of the electrical stimulation delivered to the portion of the brain may be applied in a sweeping manner. For example, the frequency of the electrical stimulation may be swept through a range of frequency values. In a frequency sweep, the frequency of the electrical stimulation may begin at one value and then may be varied, e.g., increased or decreased, from a first frequency to a second frequency. For example, electrodes 24, 26 of leads 20A and 20B, respectively, may be used to deliver electrical stimulation to patient 12 in a frequency sweeping manner while the effect of the electrical stimulation on a patient's predetermined biomarkers may be examined.

In some examples, a frequency sweep may be performed multiple times, where the patient is in a different state during each sweep. For example, a sweep may be performed while the patient is showing signs of dystonia, another while the patient is within the therapeutic window, and a third while the patient is displaying signs of dyskinesia. In some examples, other electrical stimulation parameters may be adjusted in sweeping manner. For example, stimulation amplitude, or burst frequency may also be tested. As one example, electrodes 24, 26 of leads 20A and 20B may begin delivering electrical stimulation to patient 12 being at a low frequency, which is then swept upwards. For example, electrical stimulation may be delivered in a sweeping manner from beta band frequency to a gamma band frequency (e.g., from about 30 Hz to about 140 Hz) while simultaneously monitoring LFP or EEG activity.

It should be noted that leads 20A, 20B may be separate leads, or bifurcated segments on a single lead. Some example configurations may comprise only a single lead. Two leads support bilateral stimulation in both brain hemispheres while one lead supports unilateral stimulation in one hemisphere. In some examples, one lead is positioned in or near M1 and the other lead is positioned in or near STN. In a frequency sweep, stimulation may be applied at different frequencies in a range of frequencies in a sequence, e.g., by increasing or decreasing by N Hz, where N is any number, in a linear or non-linear manner.

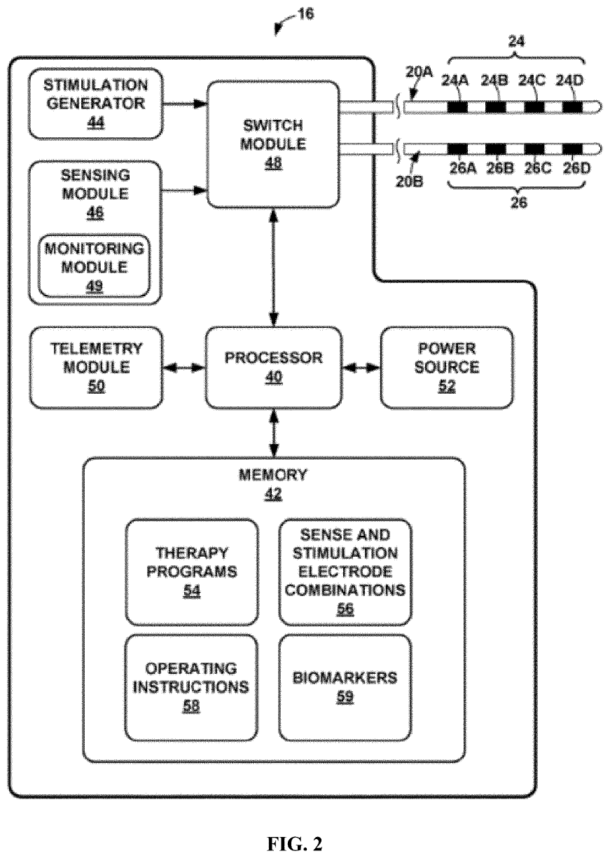

FIG. 2 is a block diagram illustrating components of an example medical device that may be used to implement the techniques of this disclosure. FIG. 2 is a functional block diagram illustrating components of an example IMD 16. In the example shown in FIG. 2, IMD 16 includes processor 40, memory 42, stimulation generator 44, sensing module 46, switch module 48, telemetry module 50, and power source 52. Memory 42 may include any volatile or non-volatile media, such as a random access memory (RAM), read only memory (ROM), non-volatile RAM (NVRAM), electrically erasable programmable ROM (EEPROM), flash memory, and the like. Memory 42 may store computer-readable instructions that, when executed by processor 40, cause IMD 16 to perform various functions.

In the example shown in FIG. 2, memory 42 stores therapy programs 54, sense electrode combinations and associated stimulation electrode combinations 56, and operating instructions 58 in separate memories within memory 42. Each stored therapy program 54 defines a particular program of therapy in terms of respective values for electrical stimulation parameters, such as a stimulation electrode combination, electrode polarity, current or voltage amplitude, pulse width, and pulse rate. In some examples, the therapy programs may be stored as a therapy group, which defines a set of therapy programs with which stimulation may be generated. The stimulation signals defined by the therapy programs of the therapy group may be delivered together on an overlapping or non-overlapping (e.g., time-interleaved) basis. In some examples, a therapy group may include a set of therapy programs wherein each of the therapy programs is associated with a different combination of biomarkers being present in a physiological signal received from the patient's brain. In some examples, a therapy group may include a combination of stimulation parameters and drug delivery parameters. The therapy groups may be store in memory 42, or another memory within IMD 16 or programmer 14. Memory 42 may also temporarily store the most recently determined biomarkers, and the therapy program currently being applied to the patient.

Sense and stimulation electrode combinations 56 stores sense electrode combinations and associated stimulation electrode combinations. As described above, in some examples, the sense and stimulation electrode combinations may include the same subset of electrodes 24, 26, or may include different subsets of electrodes. Operating instructions 58 guide general operation of IMD 16 under control of processor 40, and may include instructions for measuring the impedance of electrodes 24, 26. Processor 40 may compare received bioelectrical brain signals to values stored as biomarkers 59, as will be discussed in more detail below.

Stimulation generator 44, under the control of processor 40, generates stimulation signals for delivery to patient 12 via selected combinations of electrodes 24, 26. An example range of electrical stimulation parameters believed to be effective in DBS to manage a movement disorder of patient include the following: Frequency: between approximately 20 Hz and approximately 500 Hz, such as between approximately 50 Hz and approximately 150 Hz, or approximately 130 Hz. Voltage Amplitude: between approximately 0.1 volts and approximately 20 volts, such as between approximately 0.5 volts and approximately 10 volts, or approximately 5 volts. Current Amplitude: a current amplitude may be defined as the biological load in which the voltage is delivered. In a current-controlled system, the current amplitude, assuming a lower level impedance of approximately 500 ohms, may be between approximately 0.2 milliamps to approximately 100 milliamps, such as between approximately 1 milliamps and approximately 40 milliamps, or approximately 10 milliamps. However, in some examples, the impedance may range between about 200 ohms and about 2 kilohms. Pulse Width: between approximately 10 microseconds and approximately 5000 microseconds, such as between approximately 100 microseconds and approximately 1000 microseconds, or between approximately 180 microseconds and approximately 450 microseconds.

Stimulation generator 44 may, for example, generate either constant current-based or constant voltage-based stimulation in the form of pulses or continuous waveforms. In delivering constant current-based stimulation, stimulation generator 44 maintains the amplitude of the current at a constant level. In delivering constant voltage-based stimulation, stimulation generator 44 maintains the amplitude of the voltage at a constant level. In other examples, stimulation generator 44 may generate bipolar stimulation.

Accordingly, in some examples, stimulation generator 44 generates electrical stimulation signals in accordance with the electrical stimulation parameters noted above. Other ranges of therapy parameter values may also be useful, and may depend on the target stimulation site within patient 12, which may be within brain 28 or other portions of the nervous system. While stimulation pulses are described, stimulation signals may be of any form, such as continuous-time signals (e.g., sine waves) or the like.

Processor 40 may include any one or more of a microprocessor, a controller, a digital signal processor (DSP), an application specific integrated circuit (ASIC), a field-programmable gate array (FPGA), discrete logic circuitry, and the functions attributed to processor 40 in this disclosure may be embodied as firmware, hardware, software or any combination thereof. In some examples, the DSP may use a fast Fourier transform (FFT) algorithm. Processor 40 controls stimulation generator 44 according to therapy programs 54 stored in memory 42 to deliver, or apply, particular stimulation parameter values specified by one or more of programs, such as amplitude, pulse width, and pulse rate.

In the example shown in FIG. 2, the set of electrodes 24 includes electrodes 24A, 24B, 24C, and 24D, and the set of electrodes 26 includes electrodes 26A, 26B, 26C, and 26D. Processor 40 also controls switch module 48 to apply the stimulation signals generated by stimulation generator 44 to selected combinations of electrodes 24, 26. In particular, switch module 48 may couple stimulation signals to selected conductors within leads 20, which, in turn, deliver the stimulation signals across selected electrodes 24, 26. Switch module 48 may be a switch array, switch matrix, multiplexer, or any other type of switching module configured to selectively couple stimulation energy to selected electrodes 24, 26 and to selectively sense bioelectrical brain signals with selected electrodes 24, 26. Hence, stimulation generator 44 is coupled to electrodes 24, 26 via switch module 48 and conductors within leads 20. In some examples, however, IMD 16 does not include switch module 48.

Stimulation generator 44 may be a single channel or multi-channel stimulation generator. In particular, stimulation generator 44 may be capable of delivering a single stimulation pulse, multiple stimulation pulses or continuous signal at a given time via a single electrode combination, or multiple stimulation pulses or continuous signals at a given time via multiple electrode combinations. In some examples, however, stimulation generator 44 and switch module 48 may be configured to deliver multiple channels on a time-interleaved basis. For example, switch module 48 may serve to time divide the output of stimulation generator 44 across different electrode combinations at different times to deliver multiple programs or channels of stimulation energy to patient 12.

Sensing module 46, under the control of processor 40, may sense bioelectrical brain signals and provide the sensed bioelectrical brain signals to processor 40. Processor 40 may control switch module 48 to couple sensing module 46 to selected combinations of electrodes 24, 26, i.e., a sense electrode combination. In this way, IMD 16 is configured such that sensing module 46 may sense bioelectrical brain signals with a plurality of different sense electrode combinations. Switch module 48 may be electrically coupled to the selected electrodes 24, 26 via the conductors within the respective leads 20, which, in turn, deliver the bioelectrical brain signals sensed across the selected electrodes 24, 26 to sensing module 46. The bioelectrical brain signals may include biomarkers, e.g., amplitude and phase relationships, which are indicative of electrical activity within brain 28 of patient 12 and, in particular, electrical activity within one or more frequency bands, e.g., gamma frequency band, beta frequency band, and other frequency bands, of brain 28.

Although sensing module 46 is incorporated into a common housing with stimulation generator 44 and processor 40 in FIG. 2, in other examples, sensing module 46 may be in a separate housing from IMD 16 and may communicate with processor 40 via wired or wireless communication techniques. Example bioelectrical brain signals include, but are not limited to, a signal generated from local field potentials within one or more regions of brain 28. EEG and ECoG signals are examples of local field potentials (LFPs) that may be measured from brain 28. However, local field potentials may include a broader genus of electrical signals within brain 28 of patient 12.

Processor 40 analyzes bioelectrical brain signals in order to determine, for example, whether one or more biomarkers is present. In accordance with the techniques of this disclosure, processor 40 may select a therapy program from a plurality of therapy programs stored in memory 42, based on the presence of one or more biomarkers in the bioelectrical signals sensed by electrodes 24,26. In some examples, the programs are patient specific.

Sensing module 46 may include frequency monitoring module 49 capable of monitoring bioelectrical brain signals associated with patient 12 in selected frequency bands. Frequency monitoring module 49 may include tunable filtering and amplification capabilities that filter the bioelectrical brain signals into one or more of the beta frequency band, the gamma frequency band, and the theta frequency band, for example, and amplify the resulting filtered signal for analysis by processor 40. That is, frequency monitoring module 49 may be tuned, either by a clinician, patient, or without user intervention (i.e., automatically), to detect bioelectrical brain signals in one or more frequency bands such as the beta frequency band, or the gamma frequency band. Example circuitry capable of filtering and amplifying bioelectrical brain signals is described in U.S. Publication No. 2009/0082691 to Denison et al., entitled, “FREQUENCY SELECTIVE MONITORING OF PHYSIOLOGICAL SIGNALS,” which was published on Mar. 26, 2009, and is incorporated herein in its entirety.

In some embodiments, the bioelectrical brain signals of patient 12 may be analyzed by processor 60 of programmer 14 (or by a computer) and then transmitted via telemetry module 64 to telemetry module 50 of IMD 16.

After stimulation generator 44 delivers the electrical stimulation, or in between electrical stimulation pulses, the sensing module 46 and frequency monitoring module 49 may again monitor bioelectrical brain signals associated with patient 12. Then, processor 40 may analyze the signals to determine whether the delivered electrical stimulation resulted in modulation of one or more previously detected biomarkers. Based on the current biomarkers, processor 40 may modify the therapy being provided to patient 12. Modification may include selecting a different therapy program from memory 42, or adjusting one or more stimulation parameters.

The examples described above utilize closed-loop techniques for the delivery of electrical stimulation. That is, the examples describe sensing module 46 and frequency monitoring module 49 monitoring bioelectrical brain signals, processor 40 analyzing the bioelectrical brain signals and controlling delivery of electrical stimulation based on the analysis, sensing module 46 and frequency monitoring module 49 monitoring bioelectrical brain signals after delivery of the electrical stimulation, and processor 40 determining whether stimulation generator 44 should again deliver electrical stimulation.

Telemetry module 50 supports wireless communication between IMD 16 and an external programmer 14 or another computing device under the control of processor 40. In some examples, telemetry module 50 may support communication between IMD 16 and another medical device. Processor 40 of IMD 16 may receive, as updates to programs, values for various stimulation parameters such as amplitude and electrode combination, from programmer 14 via telemetry module 50. The updates to the therapy programs may be stored within therapy programs 54 portion of memory 42. Telemetry module 50 in IMD 16, as well as telemetry modules in other devices and systems described herein, such as programmer 14, may accomplish communication by radiofrequency (RF) communication techniques. In addition, telemetry module 50 may communicate with external medical device programmer 14 via proximal inductive interaction of IMD 16 with programmer 14. Accordingly, telemetry module 50 may send information to external programmer 14 on a continuous basis, at periodic intervals, or upon request from IMD 16 or programmer 14.

Power source 52 delivers operating power to various components of IMD 16. Power source 52 may include a small rechargeable or non-rechargeable battery and a power generation circuit to produce the operating power. Recharging may be accomplished through proximal inductive interaction between an external charger and an inductive charging coil within IMD 16. In some examples, power requirements may be small enough to allow IMD 16 to utilize patient motion and implement a kinetic energy-scavenging device to trickle charge a rechargeable battery. In other examples, traditional batteries may be used for a limited period of time.

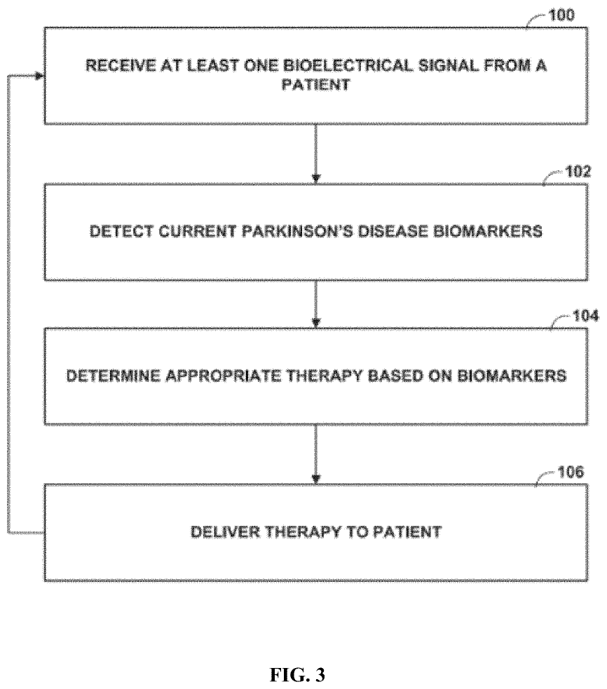

FIG. 3 is a flow chart illustrating an example method of controlling PD symptoms in a patient. Although described as being carried out within IMD 16, one or more of the steps may be completed by programmer 14. IMD 16 receives, via electrodes 24, 26, at least one bioelectrical signal from a patient (100). In some examples, electrodes 24, 26 may detect a bioelectrical signal from one or more of the motor cortex, the STN, the Zi, the GPi and the GPe. In some examples, a signal from the motor cortex may be from the M1. In some examples, electrodes 24 and 26 may detect LFPs in both the STN and motor cortex the patient's brain. Monitoring module 49 may monitor one or more frequency bands within the received bioelectrical signals. For example, monitoring module 49 may monitor both the beta and gamma bands of the detected LFPs from the patient's STN and motor cortex. Based on the received bioelectrical signals, processor 40 may detect the patient's current PD biomarkers (102). In some examples, processor 40 may determine whether a biomarker is present in the received bioelectrical signal. In some examples, processor 40 determines whether or to what degree a biomarker is present.

Processor 40 may determine an appropriate therapy based on detected current biomarkers (104). In some examples, the determination of appropriate therapy may be made without user intervention (i.e., automatically). In some examples, processor 40 may first determine a patient state based on the determined biomarkers, and select therapy parameters based on the determined patient state. In some examples, processor 40 may retrieve one or more therapy programs 54 from memory 42 based on the detected biomarkers. In some examples, the processor of programmer 14 may direct a second medical device to adjust the dosage of one or more drugs delivered to the patient. For example, programmer 14 may direct the second medical device to modify the rate of release of a medication, or the frequency of delivery of a bolus of medication. In some examples, the selection of a therapy program 54 may take into consideration when and how much drug has been delivered to the patient previously. Based on the therapy selected from memory 42 or otherwise determined by processor 40, processor 40 directs the delivery of therapy to the patient (106). As discussed above, the therapy may include stimulation at approximately 55-65 Hz, stimulation at approximately 120-140 Hz, burst stimulation, stimulation at the send frequency, delivery of a drug dosage, and/or a modification to the amount of drugs delivered. After the delivery of therapy, IMD 16 continues to monitor at least one bioelectrical signal from the patient. In this way, IMD 16 may determine the effect of the most recent therapy delivery, as well as maintain the patient within a therapeutic window in which the patient's PD symptoms are minimized along with minimal side effects.

### 3. Phase-Based Biomarker

As described further herein, embodiments of the present disclosure provide systems and methods utilizing a phase-based biomarker to identify and/or treat various diseases associated with pathological neural activity. Various neural biomarkers have been previously identified and used as diagnostic tools for assessing pathological neural activity in a subject's brain. For example, U.S. Pat. No. 8,190,251 to Molnar et al, issued May 29, 2012, incorporated herein by reference in its entirety, discloses determining biomarkers for patients with movement disorders and providing a closed-loop feedback signal to control delivery of therapy. In addition, U.S. Pat. No. 10,820,819 to Afshar et al, issued Nov. 3, 2020, incorporated herein by reference in its entirety, discloses determining biomarkers for patients with movement disorders and providing a close-loop feedback signal to control delivery of therapy. However, embodiments of the present disclosure have demonstrated that a phase metric associated with measured brain signals is advantageous as a biomarker for detecting and treating certain diseases.

In some embodiments, methods and systems of the present disclosure involve simultaneously recording signals that are bandpass filtered in a frequency range of interest. The phase is isolated from the complex signal (e.g., mathematically-complex signal) and the phase difference or phase lag between the two brain regions is calculated. The phase locking value results and the phase locking value has two components. First, the amount of coupling between the regions (how steady the phase difference is) is the phase locking value magnitude (PLV magnitude). Second the amount of lag between the two regions (in comparison to lag in the untreated state) is the phase locking value locked phase (PLV locked phase).

In some embodiments, a window around the most common phase lag is determined, and continuous periods within this phase window are considered synchronization events. In other words, detecting a synchronous event includes determining a baseline locked phase range and comparing a measured locked phase to the baseline locked phase range. From this, the total time in synchronization events, the duration of each event, and the total number of synchronous events are determined.

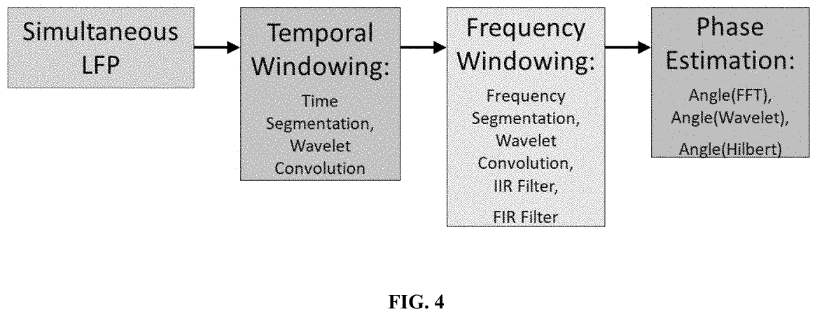

With reference to FIG. 4, to estimate the phase of LFP measurements, to preprocessing steps are implemented. The time and frequencies of interest are be windowed. In the cases of FFT and wavelet convolution both steps happen simultaneously as part of the process. In the case of the Hilbert transform, temporal and frequency windowing may happen in either order. The proposed method is agnostic to the method of phase estimation however does require reliable estimates of the phases (threshold depends on the application) and windowing around behavior/symptom relevant frequencies.

In one example method of calculating the phase metric, 500 Hz sampled local field potentials (LFP) from the STN and GP are recorded. The frequency in the beta range (13-30 Hz) with the most-reduced amplitude was determined for each STN. The LFP from the ipsilateral STN, GP and contralateral STN were all bandpass filtered in a 6 Hz band around the frequency most reduced in amplitude. A Hilbert transform is then used to calculate the analytic signal. The mean lag vector (phase locking value, PLV magnitude) was calculated (EQN. 1 and EQN. 2) as well as the locked phase (EQN. 3).

E=e^{j*(phaseI-phaseJ)} EQN. 1

PLV_{Magnitude}=|ΣE|/N EQN. 2

Locked Phase=angle(ΣE) EQN. 3

Where phaseI and phaseJ indicate the angle of the first and second local field potentials respectively and N is the number of samples in the window.

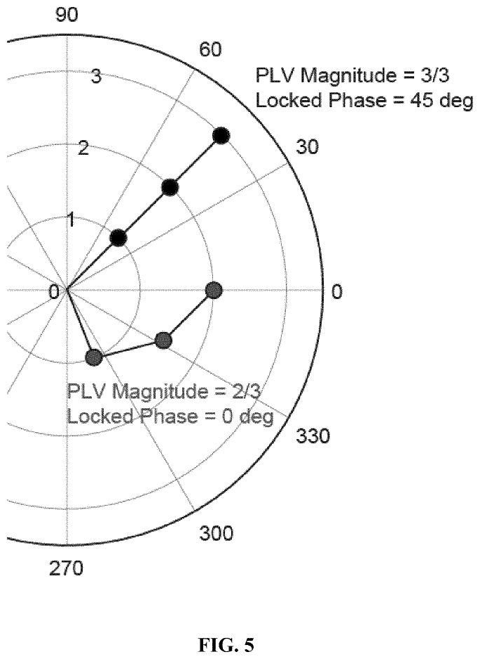

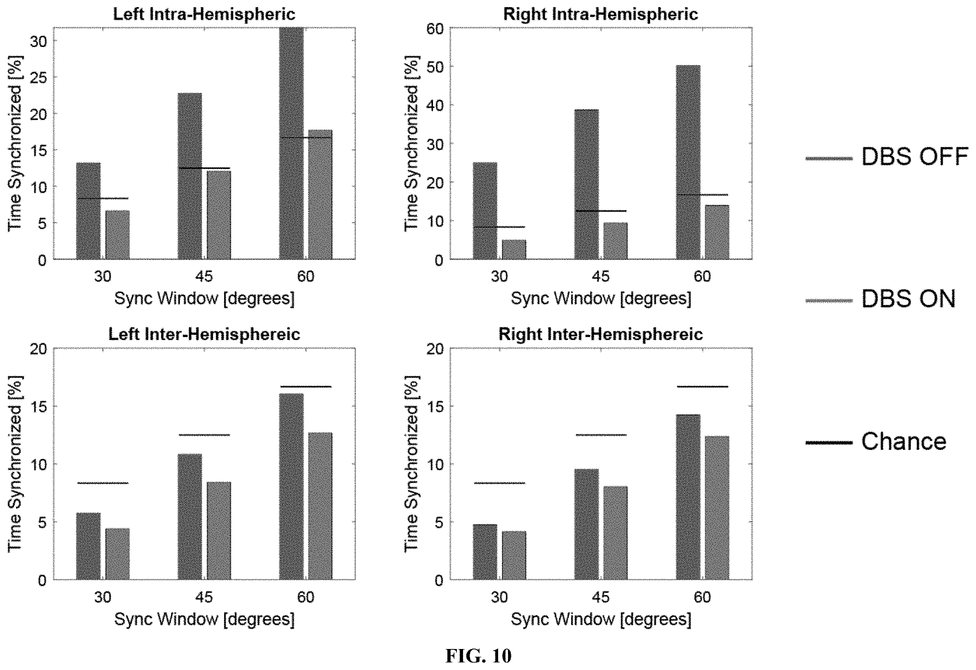

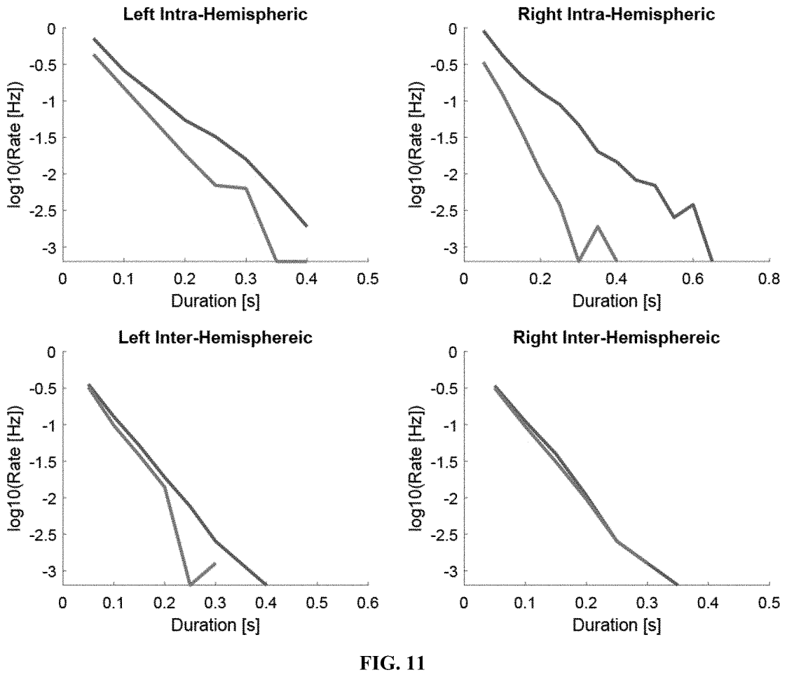

With reference to FIG. 5, example phase locking values calculations are illustrated. PLV magnitude is the sum of all phase lags divided by the number of observations. In the case of the black observations of FIG. 5, all observations are at 45 degrees therefore the PLV magnitude is a maximal 1. For the red observations of FIG. 5, the phase lags vary resulting in a PLV magnitude of ⅔ (the length of the sum is 2 divided by three observations). The locked phases are the angles from the origin to the end of the vector. As an example, all phase lags within ±45°/2 were identified as potential synchronization events (see FIGS. 10 and 11, the middle column of each panel of FIG. 10 is a 45-degree windowing for synchronous events). All events shorter than three cycles of the beta oscillation were discarded as coincidence, with the remainder considered synchronization events. The number and average duration of events were calculated within 10 second windows.

In another example method of calculating the phase metric, 500 Hz sampled local field potentials from the STN and GP of both hemispheres were filtered in the beta range (13-30 Hz). A Hilbert transform was used to calculate the analytic signal and PLV was calculated between all regions, as in EQNS. 1-3.

In some examples, to assess bradykinesia, subjects placed their hands over a 3D positioning camera (e.g., Leap Motion Controller, Ultraleap, Mountain View, Calif.). The subjects performed hand grasps with both hands for approximately 10 seconds. The grasp distance was recorded, and the grasp speed calculated as the peak-to-peak time.

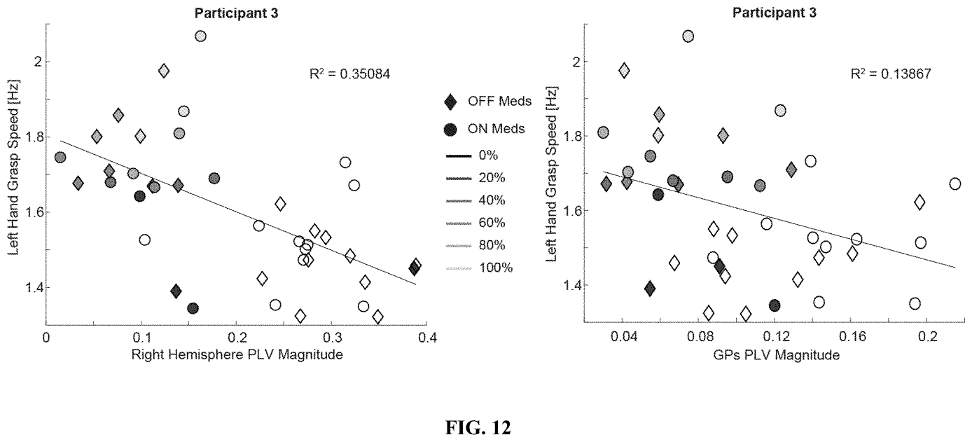

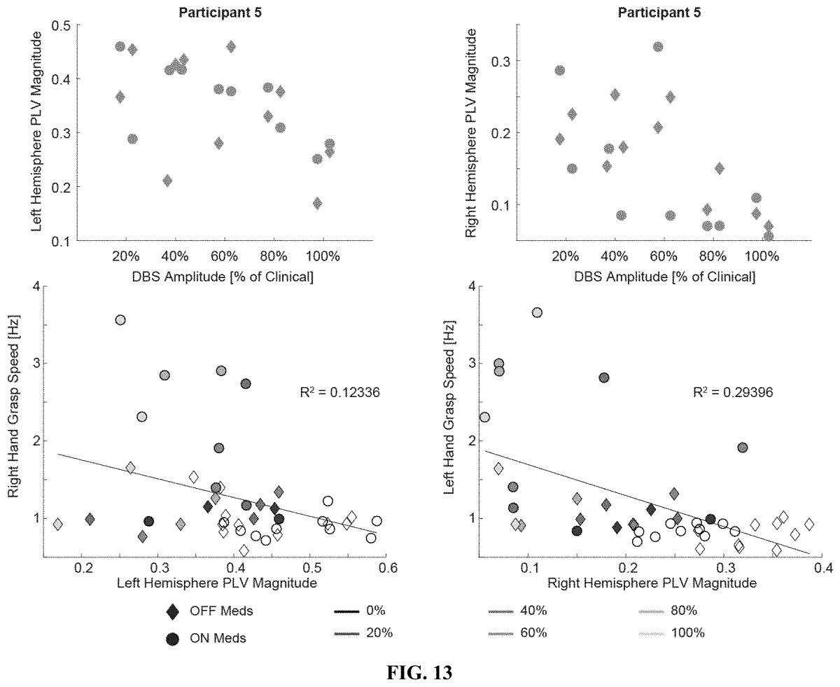

In another example method of calculating the phase matric, local field potentials were windowed (1 second) and the short term FFT taken of each channel (FIGS. 12 and 13). The average phase lag was calculated from the circular mean difference in the imaginary part of the FFT for each FFT frequency in the untreated state. Then the number of frequencies*windows outside of ±15° of the mean were summed.

The angle of the Hilbert transform, wavelet transform, Fourier transform are agnostic to the method of phase extraction. Agnostic to the source of the time series, any neural signals which have aberrant communication in disease may be used. Various utilized controllers and actuators include a bang-bang controllers and DBS, for example, or recurrent neural networks and transcranial current stimulation. In some embodiments, the biomarker is decreased, for example, STN and GP beta PLV magnitude should be decreased to reduced bradykinesia. In other embodiments, the biomarker is increased, for example, motor cortical and STN gamma PLV magnitude may be increased with effective treatment.

### 4. Methods and Systems

Embodiments of the present disclosure include a method for acquiring, with a device, a plurality of electrical signals from a brain of a subject. The plurality of electrical signals includes a first electrical signal acquired from a first brain region and a second electrical signal acquired from a second brain region. In some embodiments, the first brain region is different than the second brain region. In some embodiments, the first brain region is in a first hemisphere of the brain and the second brain region is in a second hemisphere of the brain. The method further includes determining whether a biomarker is present in the plurality of electrical signals. The biomarker described herein comprises at least one phase metric that is associated with the first electrical signal and the second electrical signal. In other words, the biomarker includes a phase metric that is based on the first and second electrical signals. The method further includes adjusting at least one therapy parameter based on the presence of the biomarker.

As described further herein, in some embodiments, the first brain region is a subthalamic nucleus, an internal segment of the globus pallidus, a motor cortex, a thalamus, an external segment of the globus pallidus, a striatum, or a cerebellum. Likewise, the second brain region is a subthalamic nucleus, an internal segment of the globus pallidus, a motor cortex, a thalamus, an external segment of the globus pallidus, a striatum, or a cerebellum. In some embodiments, the second brain region is different than the first brain region. In some embodiments, the first brain region is in a first hemisphere of the brain and the second brain region is in a second hemisphere of the brain.

As described further herein, in some embodiments, the phase metric is the phase locking value detailed herein. In some embodiments, the phase locking value includes a locked phase and a magnitude.

As described further herein, in some embodiments, the method includes determining the phase metric by temporal windowing and frequency windowing the first electrical signal and the second electrical signal. In some embodiments, the method includes determining whether the biomarker is present includes frequency isolation of the plurality of electrical signals. In some embodiments, the frequency isolation of the plurality of electrical signals includes a bandpass filter with a range of approximately 13 Hz to approximately 30 Hz. In other embodiments, the bandpass filter range is approximately 30 Hz to approximately 80 Hz. In other embodiments, the bandpass filter range is approximately 8 Hz to approximately 13 Hz. In other embodiments, the bandpass filter range is approximately 4 Hz to approximately 8 Hz. In other embodiments, the bandpass filter range is approximately 1 Hz to approximately 4 Hz.

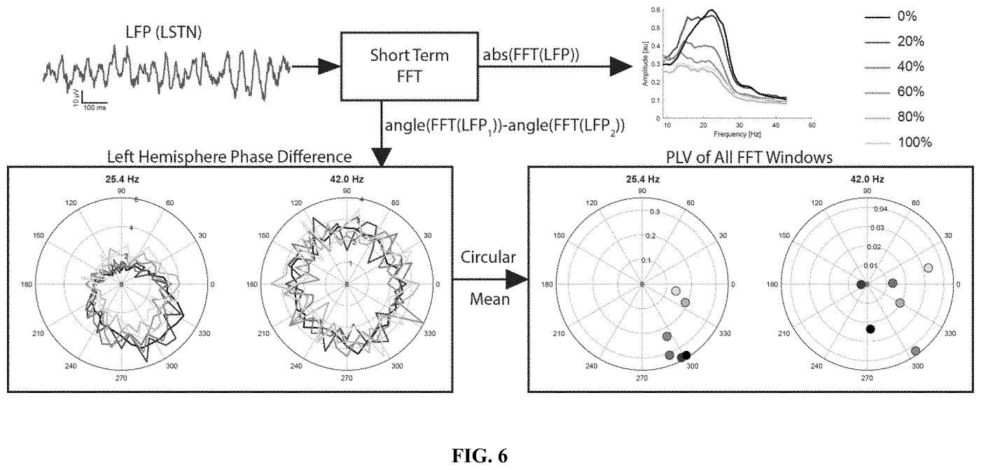

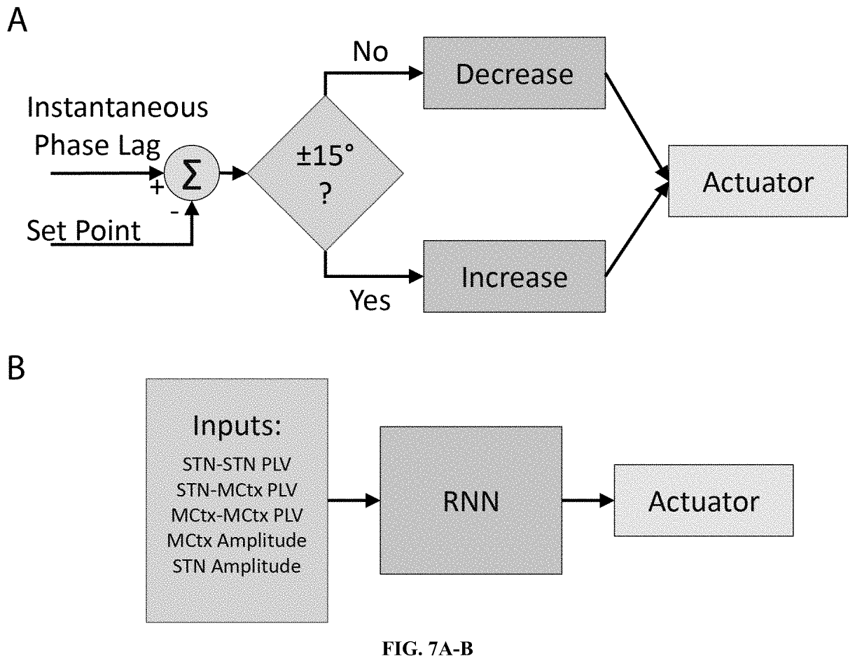

As described further herein, in some embodiments, the method includes detecting a synchronous event including determining a baseline locked phase range and comparing the locked phase to the baseline locked phase range. In some embodiments, the method includes determining a time duration of the synchronous event; and determining a total number of synchronous events. In some embodiments, the baseline locked phase range is the locked phase in the untreated (0%) state for 25.4 Hz oscillations is 300° (FIG. 6). Observations of phase lags between 285-315° (30° window) would cause an increase in DBS Amplitude (as the “increase actuator” path in FIG. 7A).

As described further herein, in some embodiments, the therapy parameter relates to an electrical stimulation to the brain by the device. In some embodiments, the therapy parameter is an amplitude of electrical stimulation, a pulse duration of the electrical stimulation, a frequency of electrical stimulation, a timing of the electrical stimulation, or a temporal pattern of the electrical stimulation. In some embodiments, the therapy parameter is a medication dose.

In some embodiments, the method includes adjusting at least one therapy parameter based on the presence of the biomarker includes closed-loop control of the biomarker. In some embodiments, the closed-loop control is a bang-bang controller, a PI controller or a PID controller. In other embodiments, the control is a neural network (e.g., a fully convoluted neural network).

Embodiments of the present disclosure also include a system comprising a device with a processor, and a plurality of electrodes in communication with the device. The plurality of electrodes are configured to be implanted in a brain of a subject. The processor is configured to acquire, via the plurality of electrodes, a plurality of electrical signals. In some embodiments, the plurality of electrodes that acquire a plurality of electrical signals is the same plurality of electrodes that provide electrical stimulation to the brain. The plurality of electrical signals includes a first electrical signal acquired from a first brain region and a second electrical signal acquired from a second brain region. The processor is also configured to determine whether a biomarker is present in the plurality of electrical signals, wherein the biomarker comprises at least one phase metric associated with the first electrical signal and the second electrical signal. The processor is also configured to adjust at least one therapy parameter based on the presence of the biomarker.

In some embodiments, the device further includes a pulse generator electrically coupled to the plurality of electrodes, wherein the pulse generator includes a power source. In some embodiments, the device is implantable.

In some embodiments, the phase metric is a phase locking value including a locked phase and a magnitude.

In some embodiments, the therapy parameter relates to an electrical stimulation to the brain delivered by the plurality of electrodes. In some embodiments, the processor adjusts the at least one therapy parameter based on closed-loop control of the biomarker.

In some embodiments, the processor is configured to detect a synchronous event by determining a baseline locked phase range and comparing the locked phase to the baseline locked phase range, and the processor is configured to detect a time duration of the synchronous event and determine a total number of synchronous events.

The electrode can be one or more electrodes configured as part of the distal end of a lead or be one or more electrodes configured as part of a leadless system to apply electrical pulses to the targeted tissue region. Electrical pulses can be supplied by a pulse generator coupled to the electrode/lead. In one embodiment, the pulse generator can be implanted in a suitable location remote from the electrode/lead (e.g., in the shoulder region); however, that the pulse generator could be placed in other regions of the body or externally to the body.

When implanted, at least a portion of the case or housing of the pulse generator can serve as a reference or return electrode. Alternatively, the lead can include a reference or return electrode (comprising a multipolar (such as bipolar) arrangement), or a separate reference or return electrode can be implanted or attached elsewhere on the body (comprising a monopolar arrangement).

The pulse generator can include stimulation generation circuitry, which can include an on-board, programmable microprocessor, which has access to and/or carries embedded code. The code expresses pre-programmed rules or algorithms under which desired electrical stimulation is generated, having desirable electrical stimulation parameters that may also be calculated by the microprocessor, and distributed to the electrode(s) on the lead. According to these programmed rules, the pulse generator directs the stimulation through the lead to the electrode(s), which serve to selectively stimulate the targeted tissue region. The code may be programmed, altered or selected by a clinician to achieve the particular physiologic response desired.

### 5. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

With reference to FIG. 6, PLV calculation is illustrated from phase of the FFT. The FFT is performed on windowed LFP traces from all leads. Phase histograms are produced by calculating the phase difference between regions for all frequencies and all windows. The PLV magnitude and locked phase are computed using EQNS. 1-3. In the case of STN-GP oscillations, 25.4 Hz exhibited high PLV magnitude without DBS and a reduction in PLV magnitude with increasing levels of stimulation. 42.0 Hz did not consistently change with increasing DBS amplitude, and therefore is unlikely to be useful for application to monitoring bradykinesia in Parkinson's disease.

### Example 2

With reference to FIGS. 7A and 7B, two possible control signals arise from this analysis: instantaneous phase lag between regions and PLV magnitude within some temporal window. These may be used with various suitable controllers and actuators with or without other controller inputs. FIG. 7A illustrates a control diagram where the instantaneous phase lag is compared to the locked phase (set point) of the untreated state. In the illustrated embodiment, if the instantaneous phase lag is within a 30-degree window of the locked phase, the stimulation amplitude is increased. FIG. 7B illustrates PLV from multiple sources are combined with amplitude measurements as inputs to a recurrent neural network controller.

### Example 3

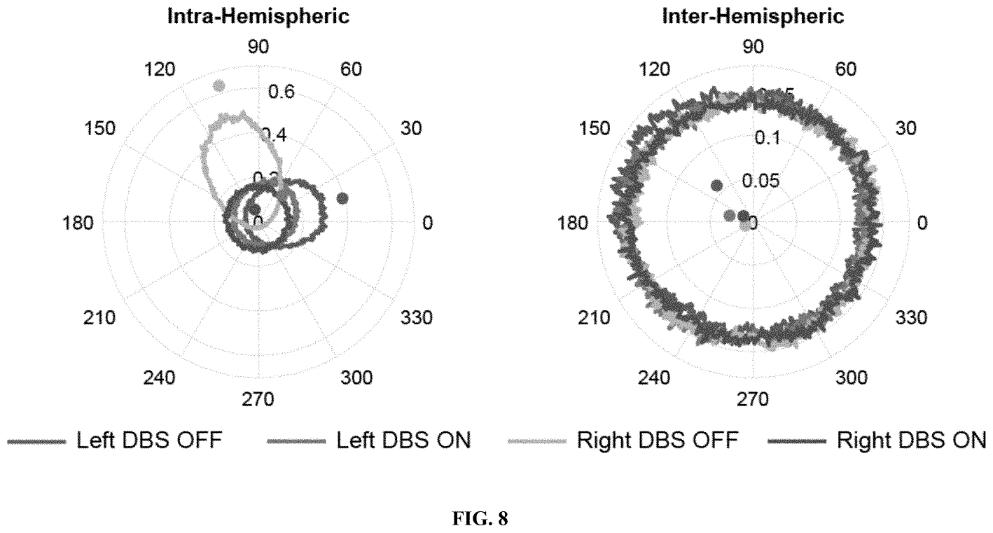

With reference to FIG. 8, radial histograms are illustrated of all phase lags between the STN and GP within a hemisphere (left) or between STNs (right). Without DBS intra-hemispheric phase locking was high. Intra-hemispheric phase locking in both hemispheres was reduced by the application of therapeutic DBS. In this participant subject, STN-STN PLV magnitude was low and remained low with DBS.

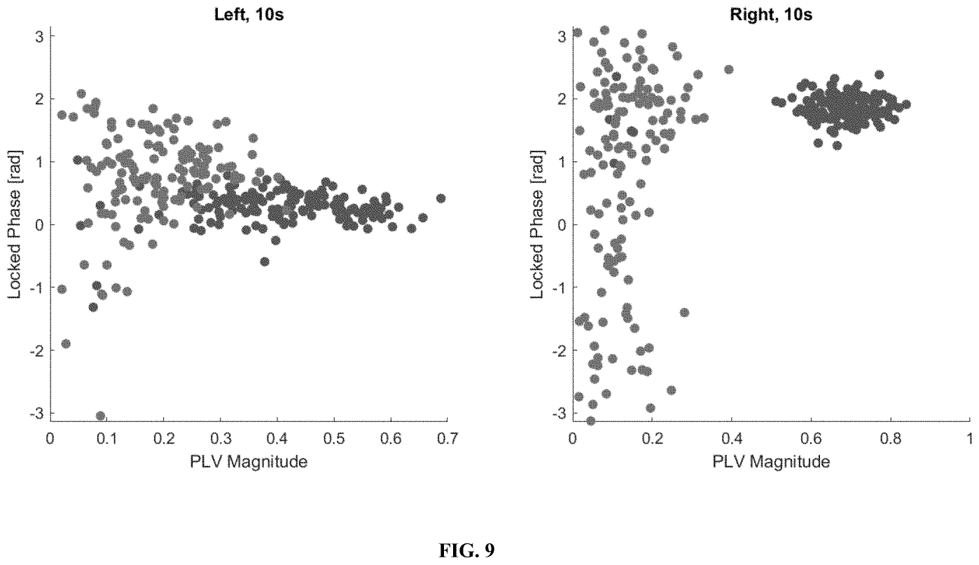

With reference to FIG. 9, intra-hemispheric PLV magnitude and locked phase in 10 second windows for untreated (blue, FIG. 9) and treated (orange, FIG. 9, therapeutic DBS) states. In the untreated state PLV magnitude is larger and a consistent phase. In the treated state the PLV is lower magnitude, and the phase is less consistent.

With reference to FIG. 10, DBS reduces total amount of time spent in synchronized events. The total time spent in synchronized events was calculated for untreated (blue, FIG. 10, DBS off) and treated (orange, FIG. 10, therapeutic DBS on) states. In all cases application of therapeutic DBS reduced time spent synchronized.

With reference to FIG. 11, DBS reduced the duration of synchronous events in the presence of pathological synchronization. The frequency of synchronous events by duration for intra-hemispheric (top) and STN-STN PLV (bottom).

### Example 4

With reference to FIG. 12, the correlation between bradykinesia and PLV magnitude is illustrated. (Left) A high degree of correlation is shown between bradykinesia (measured by hand grasp speed) and PLV magnitude between the STN and GP of the same hemisphere during the hand grasps. (Right) Additional correlation is shown between bradykinesia and interhemispheric PLV magnitude of both GPs.

### Example 5

With reference to FIG. 13, PLV magnitude is negatively correlated to DBS amplitude. (Top) intra-hemispheric PLV magnitude for each quintile of the DBS amplitude (100% corresponds to clinical value) for the left and right hemispheres, respectively. As DBS amplitude increased the PLV magnitude decreased. (Bottom) PLV magnitude-bradykinesia correlations are illustrated for the same data.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art.

What is claimed is:

1. A method comprising:
acquiring, with a device, a plurality of electrical signals from a brain of a subject, wherein the plurality of electrical signals includes a first electrical signal acquired from a first brain region and a second electrical signal acquired from a second brain region;
determining whether a biomarker is present in the plurality of electrical signals, wherein the biomarker comprises at least one phase metric associated with the first electrical signal and the second electrical signal; and
adjusting at least one therapy parameter based on the presence of the biomarker.

2. The method of claim 1, wherein the first brain region is a subthalamic nucleus, an internal segment of the globus pallidus, a motor cortex, a thalamus, an external segment of the globus pallidus, a striatum, or a cerebellum and the second brain region is a subthalamic nucleus, an internal segment of the globus pallidus, a motor cortex, a thalamus, an external segment of the globus pallidus, a striatum, or a cerebellum; wherein the second brain region is different than the first brain region.

3. The method of claim 1, wherein the phase metric is a phase locking value.

4. The method of claim 3, wherein the phase locking value includes a locked phase and a magnitude.

5. The method of claim 1, further including determining the phase metric by temporal windowing and frequency windowing the first electrical signal and the second electrical signal.

6. The method of claim 1, wherein determining whether the biomarker is present includes frequency isolation of the plurality of electrical signals.

7. The method of claim 6, wherein the frequency isolation of the plurality of electrical signals includes a bandpass filter with a range of 13 Hz to 30 Hz.

8. The method of claim 1, further including detecting a synchronous event including determining a baseline locked phase range and comparing the locked phase to the baseline locked phase range.

9. The method of claim 8, further including determining a time duration of the synchronous event; and determining a total number of synchronous events.

10. The method of claim 1, wherein the therapy parameter relates to an electrical stimulation to the brain by the device.

11. The method of claim 1, wherein the therapy parameter is an amplitude of electrical stimulation, a pulse duration of the electrical stimulation, a frequency of electrical stimulation, a timing of the electrical stimulation, or a temporal pattern of the electrical stimulation.

12. The method of claim 10, wherein adjusting at least one therapy parameter based on the presence of the biomarker includes closed-loop control of the biomarker.

13. The method of claim 1, wherein the therapy parameter is a medication dose.

14. A system comprising:
a device including a processor;
a plurality of electrodes in communication with the device, the plurality of electrodes configured to be implanted in a brain of a subject;
wherein the processor is configured to:
acquire, via the plurality of electrodes, a plurality of electrical signals, wherein the plurality of electrical signals includes a first electrical signal acquired from a first brain region and a second electrical signal acquired from a second brain region;
determine whether a biomarker is present in the plurality of electrical signals, wherein the biomarker comprises at least one phase metric associated with the first electrical signal and the second electrical signal; and
adjust at least one therapy parameter based on the presence of the biomarker.

15. The system of claim 14, wherein the device further includes a pulse generator electrically coupled to the plurality of electrodes, wherein the pulse generator includes a power source.

16. The system of claim 14, wherein the device is implantable.

17. The system of claim 14, wherein the phase metric is a phase locking value including a locked phase and a magnitude.

18. The system of claim 14, wherein the therapy parameter relates to an electrical stimulation to the brain delivered by the plurality of electrodes.

19. The system of claim 18, wherein the processor adjusts the at least one therapy parameter based on closed-loop control of the biomarker.

20. The system of claim 14, wherein the processor is configured to detect a synchronous event by determining a baseline locked phase range and comparing the locked phase to the baseline locked phase range, and the processor is configured to detect a time duration of the synchronous event and determine a total number of synchronous events.
